SEQUENCE LISTING (1 sequence)

SEQ ID NO: 1 - protein, 8 aa, Artificial Sequence
  Synthetic octapeptide
GGVLVQPG

(19) United States
(12) Patent Application Publication
(10) Pub. No.: US 2016/0349257 A1
(43) Pub. Date: Dec. 1, 2016

(54) METHODS AND COMPOSITIONS FOR THE DIAGNOSIS OF CROHN'S DISEASE

(71) Applicants: Alba Therapeutics Corporation, Baltimore, MD (US); University of Maryland, Baltimore, Baltimore, MD (US)

(72) Inventors: Julio Bai, Buenos Aires (AR); Alessio Fasano, West Friendship, MD (US); Blake Paterson, Baltimore, MD (US); Alicia Sambueli, Buenos Aires (AR)

(21) Appl. No.: 15/235,316

(22) Filed: Aug. 12, 2016

Related U.S. Application Data
(63) Continuation of application No. 14/975,110, filed on Dec. 18, 2015, now abandoned.
Application No. 14/975,110 is a continuation of application No. 13/460,917, filed on May 1, 2012, now abandoned.
Application No. 13/460,917 is a continuation of application No. 12/266,985, filed on Nov. 7, 2008, now abandoned.
Application No. 12/266,985 is a continuation-in-part of application No. 11/433,451, filed on May 15, 2006, now abandoned.
(60) Provisional application No. 60/986,272, filed on Nov. 7, 2007.
(60) Provisional application No. 60/680,868, filed on May 13, 2005.

(51) Int. Cl. G01N 33/564

(52) U.S. Cl. CPC G01N 33/564; G01N 2333/4703; G01N 2800/065; G01N 2800/56

(57) ABSTRACT

The present invention provides methods and materials, including kits, to evaluate Crohn's disease, including to diagnose, monitor, or determine the efficacy of treatment for Crohn's Disease. The methods involve determining the presence, absence, or level of zonulin in a subject sample. In certain embodiments, the need for more laborious and/or invasive tests to monitor disease state is minimized or obviated.

### PRIORITY

This application is a continuation of U.S. application Ser. No. 12/266,985, filed Nov. 7, 2008, which is a continuation-in-part of U.S. application Ser. No. 11/433,451, filed May 15, 2006 and now abandoned, which claims priority to U.S. Provisional Application No. 60/680,868, filed May 13, 2005. U.S. application Ser. No. 12/266,985 also claims priority to U.S. Provisional Application No. 60/986,272, filed Nov. 7, 2007. application Ser. Nos. 12/266,985, 11/433,451, 60/680,868, and 60/986,272 are each hereby incorporated by reference in their entireties.

The contents of the text file submitted electronically herewith are incorporated herein by reference in their entirety: A computer readable format copy of the Sequence Listing (filename: ALBA_043_02US_SeqList_ST25.txt, date recorded: Apr. 30, 2012, file size 4 kilobytes).

### FIELD OF THE INVENTION

The present invention relates to the field of diagnostics and prognostics, including the monitoring and evaluation of a condition or disease associated with an increase in gastrointestinal epithelial permeability, such as Crohn's Disease.

### BACKGROUND OF THE INVENTION

Mammalian epithelia contain structures referred to as zonula occludens (ZO) also referred to as tight junctions (TJs). These structures regulate the passage of materials through the epithelia by controlling access to the space between the epithelial cells (the paracellular pathway). To meet the many diverse physiological and pathological challenges to which epithelia are subjected, the tight junctions or zonula occludens must be capable of rapid, physiologic, reversible, transient, energy dependent, and coordinated responses that require the presence of a complex regulatory system. Examples of epithelia containing tight junctions include, but are not limited to, the intestines (particularly the small intestine), and the blood brain barrier.

In the absence of stimuli, the tight junctions are closed restricting access to the paracellular pathway. In the presence of stimuli, the tight junctions are reversibly opened. In U.S. Pat. Nos. 5,945,510 and 5,948,629, which are hereby incorporated by reference, novel mammalian proteins that function as the physiological modulator of mammalian tight junctions, have been identified and purified. These mammalian proteins function as the physiological effector of mammalian tight junctions. Certain bacteria have been shown to have toxins that stimulate the opening of tight junctions. Vibrio cholerae infected with the filamentous bacteriophage CTXΦ, produces a toxin (zonula occludens toxin, ZOT) that has been shown to cause opening of tight junctions. It has been shown that 6 His-ΔG, an N-terminal deletion of ZOT in which the first 264 amino acids have been deleted and replaced with a six histidine purification tag, retains the ability to open tight junctions.

Intestinal tight junction dysfunction may occur in certain auto-immune diseases and in a variety of clinical conditions affecting the gastrointestinal tract. Healthy, mature gut mucosa with its intact tight junction serves as the main barrier to the passage of macromolecules. During the healthy state, small quantities of immunologically active proteins cross the gut host barrier. When the integrity of the tight junction system is compromised, as with prematurity or after exposure to radiation, chemotherapy, and/or toxins, a deleterious response to environmental antigens (including autoimmune diseases and food allergies) can occur.

Crohn's disease (CD) is an inflammatory bowel disease (IBD) associated with abnormal profiles of T-cell mediated immunity, specifically an upregulated Th1 response. A number of pro-inflammatory cytokines have been implicated in development of IBD, and TNFα has been linked specifically with the pathogenesis of Crohn's disease. Indeed, anti-TNFα antibodies are used in the treatment of Crohn's disease. Presently, Crohn's disease diagnosis is based on a combination of endoscopic examination, X-rays and histologic blood and tissue tests, and after diagnosis additional tests are typically run to monitor disease status and to identify possible complications or side effects of medication. This procedure is highly invasive and entails the risk of placing the subject under anesthesia. It would be useful to have a less invasive method of determining if a subject had Crohn's disease and/or to monitor the status of the disease and/or the efficacy of treatment. This need and others are met by the present invention.

### SUMMARY OF THE INVENTION

The present invention provides materials and methods for diagnosing Crohn's disease in a subject, including methods for evaluating Crohn's disease, and evaluating the status of Crohn's disease, before, during, and after treatment. Such methods may comprise obtaining a patient or subject sample and determining the presence or absence or level of zonulin in the sample. The level of zonulin in the sample may be determined with respect to control levels, such as with respect to non-disease samples (e.g., normal samples from subjects not afflicted with Crohn's Disease), or with respect to pre-treatment zonulin levels or prior tested zonulin levels for the patient.

The detection of zonulin in the sample, e.g., the level of zonulin in the sample, is predictive or indicative of the presence or severity of active Crohn's disease. In certain embodiments, the level of zonulin in the patient sample is correlative with the active status or severity of Crohn's Disease in a patient previously diagnosed or undergoing treatment for Crohn's disease. The absence of detectable zonulin (or a decrease in zonulin levels as compared to controls) in the sample is predictive of the absence of active Crohn's disease, or a quiescent state of the disease. In some embodiments, a decrease in zonulin levels upon treatment for Crohn's Disease, as compared to prior or pretreatment zonulin levels, is correlative with an effective treatment or disease remission.

Thus, in certain embodiments, the invention provides a method for monitoring

Crohn's Disease in a Crohn's Disease patient. The method comprises monitoring the level of zonulin in patient samples, as described herein. Zonulin levels may be determined on a periodic basis to monitor the status of the disease and/or the efficacy of treatment. In some embodiments, Crohn's Disease is monitored, e.g., by determining zonulin levels, without the use or need for laborious or invasive examinations, such as endoscopic examination, histologic blood or tissue tests, or X-ray. In various embodiments, zonulin levels may be determined weekly, monthly, or from about one to about ten times per year to monitor the disease.

In certain embodiments, the invention involves determining zonulin level(s) in a sample from a patient suspected of having Crohn's Disease. For example, the patient may have one or more symptoms associated with Crohn's Disease, such as abdominal pain, diarrhea (which may be visibly bloody), vomiting, or weight loss. In such embodiments, the level of zonulin in patient sample(s) is further indicative of the presence of Crohn's Disease or active Crohn's Disease. The level of zonulin in the patient's sample may then negate the need for, or otherwise warrant, more invasive or expensive procedures to diagnose the presence of Crohn's Disease. For example, where zonulin levels are significantly elevated as compared to control values, the presence of Crohn's Disease may be further confirmed by additional diagnostic tests for Crohn's disease, such as one or more of endoscopic examination, X-ray (including CAT), histologic blood or tissue test, or other test known in the art.

In certain embodiments, the invention involves determining zonulin level(s) in a sample from a patient diagnosed with Crohn's Disease. For example, the patient may have been diagnosed with Crohn's Disease by one or more of endoscopic examination, X-ray (including, for example, CAT), histologic blood or tissue test, or other test commonly used to diagnose the disease. In some embodiments, zonulin levels are determined periodically to monitor the disease, such as to determine whether the disease is active or in remission, or to determine the severity of the disease state. Zonulin levels may be determined on a periodic basis, e.g., at a frequency as described above, and without the need for invasive or costly procedures. In other embodiments, zonulin levels are determined to select appropriate treatment. For example, where the Crohn's Disease patient has elevated levels of zonulin, e.g., as described herein, the patient may be treated with an inhibitor of gastrointestinal permeability (e.g., AT1001).

In still other embodiments, the invention involves determining zonulin level(s) in a sample from a Crohn's Disease patient that is undergoing treatment for the disease. For example, the patient may be undergoing treatment with one or more of an antiinflammatory agent, a corticosteroid, a topical antibiotic, an immunomodulator, or an inhibitor of epithelial tight junction permeability. Exemplary antiinflammatory agents may include ASA compounds such as sulfasalazine (Azulfidine) and mesalamine (Pentasa, Asacol, Dipentum, Colazal, Rowasa enema, Canasa suppository), which act via direct contact (topically) with the inflamed tissue. The patient may be undergoing treatment with a corticosteroid that acts systemically to decrease inflammation throughout the body, or otherwise a local or topical corticosteroid (for example, budesonide) that acts via direct contact with the inflamed tissue. Exemplary antibiotics include metronidazole (Flagyl) and ciprofloxacin (Cipro). In certain embodiments, the patient is undergoing treatment with an agent that prevents or reduces the permeability of epithelial tight junctions in the gastrointestinal tract. Such agents, including the peptide Gly-Gly-Val-Leu-Val-Gln-Pro-Gly (SEQ ID NO: 1) which is also referred to as AT1001, are described in U.S. Pat. No. 6,458,925 and US 2007/0196501, both of which are hereby incorporated by reference in their entireties.

Any type of sample known to those skilled in the art may be obtained and analyzed for the presence or absence or level of zonulin. Generally, the sample is suitable for determining levels of zonulin, and particularly for determining levels of zonulin that are indicative of tight junction permeability in the gastrointestinal tract. In certain embodiments, the sample obtained from the patient or subject is a blood sample or a urine sample. In some embodiments, serum may be isolated from the blood sample and the presence or absence or level of zonulin may be determined in the serum. Patient samples, including blood and serum samples (as well as other samples derived or fractionated from blood), may be isolated by known techniques.

The invention involves determining the presence or absence or level of zonulin in patient samples, as described above. The presence or absence or level of zonulin may be determined using any technique for determining protein levels, including immunoreactivity with an antibody that recognizes zonulin. For example, in some embodiments, determining the presence or absence or level of zonulin in a sample may comprise contacting the sample with an antibody that binds zonulin, under binding conditions, to create a binding complex when zonulin is present, and then determining the presence, absence, or amount of the binding complex, e.g., amount relative to a control. For example, the invention may involve a sandwich immunoassay with a first and second antibody, where the first antibody binds to zonulin under the binding conditions, and the second antibody binds zonulin under the binding conditions. The first antibody may be immobilized or trapped on a support, and the second antibody may comprise a detection means to thereby allow detection of bound zonulin/antibody complex. The first and/or second antiobodies in some embodiments are monoclonal. Detection means may include a dye, a fluorescent label, a radiolabel, an enzymatic label, a colloidal gold label, or other detection means, and may be present on the second antibody, or an antibody reactive with the second antibody or a moiety thereof (e.g., biotin). Equivalent immunoassay formats are known, including competition assay formats, and are within the scope of the invention.

In some embodiments, at least one antibody may be raised against a protein comprising a fragment of zonula occludens toxin, such as the ΔG fragment of the zonula occludens toxin. In one particular embodiment, the first antibody may be raised against a protein comprising a fragment of zonula occludens toxin, for example, the ΔG fragment of zonula occludens toxin. In another embodiment, the second antibody may be raised against a protein comprising zonula occludens toxin, such as the ΔG fragment of the zonula occludens toxin. Typically, the second antibody may comprise a detectable moiety as described above.

The present invention further provides kits for diagnosing or monitoring Crohn's Disease. The kits of the invention comprise components for detecting zonulin, and may employ any assay format, including microtiter or strip assay formats that are convenient and known in the art. Alternatively, the kit may comprise one or more containers containing one or more antibodies. For example, a kit of the invention may comprise a first container containing the first antibody and a second container containing the second antibody. First and second antibodies are described above. Any antibody capable of binding zonulin may be used as a first or second antibody. Typically, the first and second antibodies bind at different regions of zonulin such that both a first and a second antibody may be bound to zonulin at the same time, to thereby form a binding complex in the presence of zonulin.

In some embodiments, kits of the invention may comprise one or more antibodies wherein at least one antibody was raised against a protein comprising a fragment of zonula occludens toxin, or in some embodiments a protein comprising the ΔG fragment of zonula occludens toxin. The at least one antibody may be supplied in an immobilized form, e.g., on a solid support, or otherwise in a container or vial. A second antibody may be supplied for detecting antibody/zonulin binding complex. The antibody may be supplied in any suitable form, such as a separate container, but may be operably supplied as part of a self-contained assay format, such as a filter or strip assay. The second antibody may further comprise a detectable moiety as described.

Kits of the invention may also comprise one or more additional containers containing one or more additional reagents. Additional reagents include salts, buffers, metal ions, enzyme substrates and the like. In some embodiments, kits of the invention may comprise a container containing ΔG fragment of zonula occludens toxin. In some embodiments, kits of the invention may comprise a container containing zonulin.

The kits of the invention may be supplied as a companion diagnostic, for example, together with an agent for treating Crohn's Disease. Such agents include AT1001, described in U.S. Pat. No. 6,458,925 and US 2007/0196501, both of which are hereby incorporated by reference in their entireties. Thus, the kit enables selection of appropriate treatment for Crohn's Disease patients.

### BRIEF DESCRIPTION OF THE DRAWINGS

FIG. 1 shows zonulin levels measured in samples obtained from treatment naïve Crohn's patients with active disease (n=26); mesalamine treated Crohn's patients with active disease (n=19); and mesalamine treated Crohn's patients in remission (n=14).

### DETAILED DESCRIPTION OF THE INVENTION

As used herein a subject is any animal, e.g., mammal, that may suffer from

Crohn's Disease or which may be treated for Crohn's Disease. Subjects include, but are not limited to, humans. Thus, the patient may be a human or veterinary patient. As described, the patient may be suspected of having Crohn's Disease, e.g., based upon the existence of one or more symptoms of Crohn's Disease, or may be previously diagnosed as having Crohn's Disease, for example, based upon tests and examinations known in the art or described herein. Alternatively, the patient may be undergoing treatment for Crohn's Disease.

As used herein, antibodies are named using the prefix anti-followed by the antigen to which the antibody binds. Thus, for example, anti-zonulin antibody is an antibody that binds to zonulin. Antibodies may be monoclonal or polyclonal, or may be functional antibody fragments.

As used herein, zonulin refers to one or more proteins or factors that modulate, affect, or induce, directly or indirectly, the opening of mammalian tight junctions of the gastrointestinal tract. In certain embodiments, zonulin is the detectable component of a sample (e.g., blood or blood product) antigenically related to the ΔG fragment of zonula occludens toxin. In such embodiments, zonulin is specifically recognized by an antibody raised against the ΔG fragment.

As discussed above, in various embodiments, the present invention provides materials and methods for diagnosing Crohn's Disease. Further, the present invention provides materials and methods for assessing the status of patients suffering from Crohn's Disease and for monitoring the efficacy of treatment of patients suffering form Crohn's Disease. In certain embodiments, the Crohn's Disease is Crohn's enteritis, which is generally confined to the small intestine (the first part, called the jejunum or the second part, called the ileum). The Crohn's Disease may involve the ileum alone (“Crohn's ileitis”). In certain embodiments, the Crohn's Disease is Crohn's terminal ileitis, which refers to inflammation that affects only the very end of the small intestine (terminal ileum), the part of the small intestine closest to the colon.

The measurement of zonulin can be accomplished by any means appropriate. Typically, however, such measurement will be accomplished by immunoassay, that is, by determining the binding of an anti-zonulin antibody to zonulin in the sample under assay. As is well-known in the art, the determination of such antibody binding can be performed using a great variety of immunoassay formats including that described above and in Example 1.

The present invention is not limited to any particular immunoassay format. Preferred, however, will be heterogeneous immunoassay formats such as sandwich immunoassay formats in which the antigen of interest is detected by formation of a “sandwich” complex of a separation antibody and a detection antibody. The separation antibody (first antibody) is immobilized or immobilizable such as to a solid support, e.g., the walls of a microtiter plate, a filter, a latex particle, a macrobead, and the like. The detection antibody (second antibody) is labeled or labelable, directly or indirectly, with a detectable label such as, without limitation, enzymes or enzyme cofactors or substrates, chemiluminescent or fluorescent molecules, radioisotopes, dyes, and the like. The manner of detection can be any means conventionally associated with the particular label employed, e.g., a spectrophotometer in the case of enzymes or enzyme cofactors or substrates, a luminometer or fluorometer in the case of chemiluminescent or fluorescent molecules, or beta or gamma counters in the case of radioisotopes. Alternatively, the detection antibody can be detected by means of a labeled isotypic antibody.

In certain embodiments, the level of zonulin in the sample is compared to a control level, such as the level indicative of a normal patient (that is, an individual not afflicted with Crohn's Disease). In such embodiments, a measurement indicative of Crohn's Disease is significantly higher (e.g., statistically significant) than the control level. For example, a sample that tests positive for Crohn's Disease may be at least 20%, 50%, 75%, or at least 100% higher than the control level. In certain embodiments, a sample that tests positive for active Crohn's Disease is from 2 to 10 times (e.g., at least two times, three times, four times, or five times) the level of a normal control.

In certain other embodiments, the level of zonulin is compared to levels determined in samples from the same patient, for example, taken prior to or during treatment for Crohn's Disease. In such embodiments, a measurement indicative of controlled or quiescent Crohn's Disease is significantly lower (e.g., statistically significant) than a prior or pretreatment level. Statistically significant levels that reflect controlled disease may be 75%, 50%, 20%, 10% of the prior or pretreatment level (e.g., the active disease level). In certain embodiments, a sample reflective of controlled disease is from about ½ to 1/10 (e.g., about ⅓, ¼, or ⅕) the level of the prior or pretreatment level.

The methods and compositions of the present invention can be used with other methods and compositions in use for the diagnosis and treatment of Crohn's disease. Such methods and compositions include endoscopic examination, X-rays and histologic blood and tissue tests, as well as additional tests that may be run to monitor disease status and to identify possible complications or side effects of medications. In certain embodiments, the need for such tests to confirm Crohn's Disease or to monitor disease state is minimized or obviated.

Endoscopic methods which may be used in combination with the methods and compositions of the present invention include, but are not limited to, sigmoidoscopy, colonoscopy, and endoscopic ultrasound.

Radiologic methods which may be used in combination with the methods and compositions of the present invention include, but are not limited to, X-rays without contrast, X-rays using liquid contrast agents, including barium, computerized tomography (CT) scanning, and leukocyte, scintigraphy.

Histologic blood and tissue tests which may be used in combination with the methods and compositions of the present invention include, but are not limited to, a complete blood count (CBC), measurement of C-reactive protein, and tests to monitor for side effects of certain medications, including for example, liver function tests and stool studies can identify treatable bacterial infections.

The invention will now be further described with reference to the following non-limiting examples:

### Example 1

Analysis of zonulin levels in Crohn's disease patients categorized as being in remission or active according to Modigliani's endoscopic critria (Modigliani et al, Gut 1989, 30, 983-989).

Only 29% of patients in clinical remission also achieve endoscopic remission in cases using steroids (Modigliani R Gastroenterology 1990; 98:811-818) according to a standard where endoscopic remission was defined as no lesion at all or scarred lesion only (Landi B 1992; 102: 1647-1653). The Modiglani Index for assessment of endoscopic activity is the criterion most widely validated and used in several trials. In that index, activity only takes into account frank erythema and frank swollen mucosa. It must be considered that the index specifies that both slight or moderate erythema or slight or moderate mucosal swelling should not be considered features of active disease.

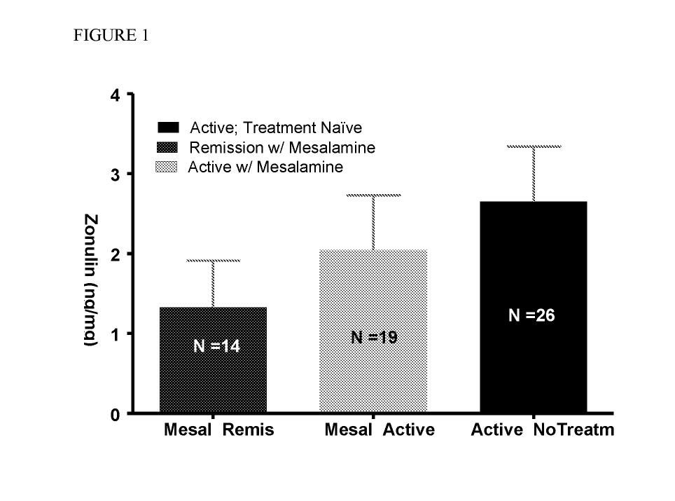

According to the Modigliani Index, individual data of several patients suggested zonulin as a marker for disease activity, disease location or disease pattern in Crohn's disease. According to this hypothesis zonulin could be an early finding in Crohn's disease, and this could support the findings of endoscopic examination and might be a suitable criterion to define remission, since mucosal lesion precedes appearance of clinical symptoms. The correlation between measured zonulin levels and disease activity according to the Modigliani Index is set forth in Table 1 and FIG. 1.

TABLE 1
Zonulin levels in Crohn's disease patients

| | Mesal Remis | Mesal Active | Active Naïve |
|---|---|---|---|
| Geometric Mean | 0.1827 | 0.3850 | 0.8193 |
| Lower 95% of Geom. Mean | 0.03476 | 0.1038 | 0.3334 |
| Upper 95% of Geom. Mean | 0.9606 | 1.428 | 2.014 |

Serum zonulin measurement by sandwich enzyme-linked immunosorbent assay. Zonulin sandwich enzyme-linked immunosorbent assay (ELISA) was performed as described in El Azmar et al. Gastroenterology 123:1607-1615, 2002 with minor modifications.

Briefly, plastic microtiter plates (Costar, Cambridge, Mass.) were coated with rabbit zonulin cross-reacting anti-Zonula occludens toxin (Zot) derivative ΔG IgG antibodies (10 μg/ml in 0.1 mol/l sodium carbonate buffer, pH 9.0). These antibodies were prepared by immunizing a rabbit with ΔG using standard protocols.

After overnight incubation at 4° C., plates were washed four times in Tris buffered saline 0.05% Tween 20 (TBS-T) and blocked by incubation for 1 h at 37° C. with TBS-T. After four TBS-T washes, five ΔG serial standards (50, 25, 12.5, 6.2, 3.1, and 0 ng/ml) and patient sera samples (1:101 dilution in TBS-T) were added and incubated overnight at 4° C. After four washes with Tris buffered saline 0.2% Tween 20 buffer, plates were incubated with biotinylated anti-Zot IgG antibodies (U.S. Pat. No. 5,945,510) for 4 h at 4° C. and contacted with streptavidin-conjugated alkaline phosphatase. A color reaction was developed by using a commercial kit (ELISA amplification kit; Invitrogen). The absorbance at 495 nm was measured with a microplate auto-reader (Molecular Devices Thermomax Microplate Reader).

While the invention has been described in detail, and with reference to specific embodiments thereof, it will be apparent to one of ordinary skill in the art that various changes and modifications can be made therein without departing from the spirit and scope thereof, and such changes and modifications may be practiced within the scope of the appended claims. All patents and publications herein are incorporated by reference to the same extent as if each individual publication was specifically and individually indicated to be incorporated by reference in their entirety.

What is claimed is:

1. A method of evaluating Crohn's disease in a subject, comprising:
obtaining a subject sample; and
determining the level of zonulin in the sample, wherein an increased level of zonulin is predictive of active Crohn's disease.

2. The method of claim 1, wherein the subject has one or more symptoms of Crohn's Disease.

3. The method of claim 1 or 2, wherein the subject has been diagnosed with Crohn's Disease.

4. The method of any one of claims 1 to 3, wherein the subject is undergoing treatment for Crohn's Disease.

5. The method of any one of claims 1 to 4, wherein the sample is a blood sample.

6. The method of claim 5, further comprising:
isolating serum from the sample.

7. The method of claim 2, further comprising performing an endoscopic examination.

8. The method of claim 2, further comprising performing a histologic blood test.

9. The method of any one of claims 1 to 8, wherein determining the level of zonulin comprises:
contacting the sample with a first antibody that binds to zonulin under binding conditions;
contacting the bound sample with a second antibody that binds zonulin under binding conditions; and
detecting the presence of bound second antibody.

10. The method of claim 9, wherein one antibody was raised against a protein comprising a fragment of zonula occludens toxin.

11. The method of claim 10, wherein the one antibody was raised against a protein comprising ΔG fragment of zonula occludens toxin.

12. The method of claim 9, wherein the first antibody was raised against a protein comprising a fragment of zonula occludens toxin.

13. The method of claim 9, wherein the first antibody was raised against a protein comprising ΔG fragment of zonula occludens toxin.

14. The method of claim 9, wherein the second antibody was raised against a protein comprising ΔG fragment of zonula occludens toxin.

15. The method of claim 9, wherein the second antibody comprises a detectable moiety.

16. The method of claim 15, wherein the detectable moiety is biotin.

17. The method of any one of claims 1 to 16, wherein the method is performed on a periodic basis to monitor disease state.

18. A kit for diagnosing Crohn's disease, comprising:
a means for binding zonulin; and
a means for detecting zonulin.

19. The kit of claim 18, wherein the means for binding zonulin comprises:
a first container containing a first antibody.

20. The kit of claim 18, wherein the means for detecting zonulin comprises:
a second container containing a second antibody.

21. The kit of claim 18, further comprising the ΔG fragment of zonula occludens toxin.

22. A method of evaluating Crohn's disease in a subject diagnosed as having Crohn's disease, consisting essentially of:
obtaining a subject sample; and
determining the level of zonulin in the sample, wherein an increased level of zonulin is predictive of active Crohn's Disease or severity of Crohn's Disease.
